(19) United States
(12) Patent Application Publication
(10) Pub. No.: US 2011/0003000 A1
(43) Pub. Date: Jan. 6, 2011

(54) Transvaginal Delivery of Drugs

(75) Inventors: Gerianne Tringali DiPiano, Malvern, PA (US); Thomas I. Janicki, Shaker Heights, OH (US); Peter Kevin Mays, Philadelphia, PA (US); John Andrew Ziemniak, Gwynedd Valley, PA (US)

Correspondence Address: Pabst Patent Group LLP, 1545 PEACHTREE STREET NE, SUITE 320, ATLANTA, GA, 30309, US

(73) Assignee: FemmePharma Holding Company, Inc.

(21) Appl. No.: 12/497,865

(22) Filed: Jul. 6, 2009

(51) Int. Cl. A61K 9/14; A61K 9/12; A61K 31/56; A61K 31/24; A61P 13/00; A61P 15/02

(52) U.S. Cl. 424/489; 424/45; 514/169; 514/534

(57) ABSTRACT

Drug delivery compositions which are suitable for transvaginal administration for the treatment of diseases and disorders of the urogenital tract are described. The drug delivery compositions are administered directly to the vagina using a convenient transvaginal application that deposits a very small volume of drug at the desired site for delivery. This method of administration reduces the systemic levels of the drugs and decreases the side effects which are associated with systemic administration. In the preferred embodiment, the compositions are in the form of a gel. The formulation is administered in volumes of less than or equal to 1 milliliter. In the preferred embodiment, the composition contains an antimuscarinc drug, such as oxybutynin.

### BACKGROUND OF THE INVENTION

The present invention relates to pharmaceutical preparations for the treatment of diseases and disorders of the urinogenital tract.

Diseases and disorders of the bladder and urinary tract include cancers, infections, urinary incontinence, urethral syndrome, urethritis, female sexual dysfunction, and interstitial cystitis. These diseases and disorders may be treated by a variety of systemically administered pharmaceutical preparations. However, systemic delivery results in a number of side effects. For example, when oxybutynin hydrochloride is administered orally for the treatment of urinary incontinence, side effects including dizziness, blurred vision, dry mouth, and cardiovascular manifestations are experienced by patients. These side effects often limit the use of the formulation by the patients.

Local application of drugs for the treatment of bladder disorders, such as urinary incontinence, bladder cancer, and interstitial cystitis has been described in the literature. Intravesical administration of doxorubicin for bladder cancer, DMSO for interstitial cystitis, and oxybutynin hydrochloride for urinary incontinence have been shown to provide relief from symptoms without the side effects observed during systemic therapy. However intravesical administration is inconvenient and requires a trained technician to administer the drug formulation. Further, inserting and removing a catheter from a patient increases the risk of patient infection.

Urinary incontinence is defined by the International Continence Society as the “involuntary loss of urine that represents a hygienic or social problem to the individual.” Many physicians classify female urinary incontinence into either stress or urge incontinence. Urge incontinence is an involuntary loss of urine associated with an abrupt and strong desire to void. Often urge incontinence is associated with detrusor overactivity. Stress incontinence is an involuntary loss of urine during coughing, sneezing, laughing, or other physical activities that increase abdominal pressure. Urinary incontinence affects approximately 13 million American women. Urge urinary incontinence occurs more frequently in women than in men. Next to stress incontinence, urge incontinence is the second most common cause of female urinary incontinence. It has been estimated that two-thirds of women wear some protection (i.e., pads) if incontinence occurs at least twice weekly.

Bladder emptying requires the maintenance of pressure in the bladder during the tonic phase of the contractile response. Bladder emptying and continence also depend upon the tonic phase of the contractile responses of the urethra. Thus, a defect in the ability of the bladder to sustain a contraction reduces the ability of the bladder to empty. Similarly, in women, if the urethra is unable to sustain increased tension during bladder filling, stress incontinence may result. Women depend upon urethral smooth muscle tension for continence to a much greater extent than men, where the prostate and better developed external sphincter add significantly to urethral tension and continence.

A second common cause of urinary incontinence is when the bladder contracts during bladder filling, i.e. hyperreflexia. These contractions are primarily due to neurogenic mechanisms involving the release of acetylcholine (herein referred to as “Ach”) and muscarinic-mediated bladder contractions.

One agent that has proven to be clinically effective in the treatment of urinary incontinence is oxybutynin. Oxybutynin relaxes the bladder by muscarinic inhibition and by direct relaxation of smooth muscle. Oxybutynin is one of the most widely prescribed oral medications for the treatment of stress incontinence (also referred to as “bladder instability”) and urge incontinence (see Anderson, et al., J. Urol., 161: 1809-1812 (1999); Gupta & Sathyan, J. Clin. Pharmacol., 39: 289-296 (1999); and Thütiroff, et al., J. Urol., 145: 813-816 (1991)). However, its major disadvantages include its relatively short half-life and the resulting anticholinergic side effects. In many cases, patients do not follow their prescribed treatments due to the frequent dosing schedule and the side effects. In order to improve patient compliance, a long-acting preparation (once a day) was developed and tested (Goldenberg, Clin Ther, 21: 634-642 (1999); Anderson, et al., J Urol., 161: 1809-1812 (1999); and Versi, et al., Obstet. Gynecol., 95: 718-721 (2000)). Results of these studies demonstrate that the time-released preparation is as effective as the original preparation, but also produces the side effects associated with the administration of the original formulation (Versi, et al., Obstet. Gynecol., 95: 718-721 (2000) and Corner & Goa, Drugs Aging, 16: 149-155 (2000)).

Intravesical instillation of oxybutynin has been evaluated. Brendler describes the intravesical administration of oxybutynin chloride for the treatment of dysfunctional bladders in a study of eleven patients with persistent urge incontinence and frequent side effects from the use of oral anticholinergic agents (Brendler et al., J Urology, 141(6): 1350-52 (June, 1989)). Ten out of eleven patients reported improvement and became totally continent, and no side effects were observed. Similarly, Saito describes using a catheter to deliver an oxybutynin solution to patients suffering from urinary incontinence. (Saito et al., Neurology and Urodynamics, 19: 683-88 (2000)) This method was effective, and the patients did not experience side effects.

Although this method of treatment can avoid the first pass metabolism and reduce systemic side effects (Buyse, et al., J. Urol., 160: 892-896 (1998); Masad, et al., J. Urol., 148: 595-597 (1992)), it is inconvenient and does not provide a method for continuous delivery, as discussed above.

A few researchers have begun to investigate transvaginal delivery of anticholinergics, such as oxybutynin and propantheline bromide, to the bladder. Geraghty, et al., Pharmaceutical Research, 13(8):1265-1271 (1996) disclose a formulation containing monoolein and an antimuscarinic drug, either oxybutynin hydrochloride or propantheline bromide. Monoolein is a polar lipid which forms gels in the presence of water. Geraghty performed in vitro experiments to determine if the gel was an effective delivery system for the antimuscarinic drugs. The gel formulations demonstrated a sustained release of the antimuscarinic drugs for approximately 18 hours. Based on the release profile, it appeared that the drug diffused out of the gel. However, such results are not predictive of what would happen in vivo since the gel could degrade or the drug could be delivered systemically.

Schröder, et al., Urology, 56 (6): 1063-1067 (2000) describes inserting a solid device which contained oxybutynin in the vagina of a rabbit. Though Schröder's insert was effective at reducing the systemic levels of oxybutynin, inserts are often uncomfortable for patients. Although offering advantages over the oral route in respect of avoiding some unwanted side effects, semi-solid vaginal preparations nevertheless suffer from several disadvantages, such as leakage (influenced in part by large volume of material typically administered intravaginally) with resulting low residence time and messiness which contribute to poor subject or patient. For example Barnhart et al. Human Reprod., 16:1151-1154 (2001), using magnetic resonance imaging to visualize vaginal distribution of 5 ml of nonoxynol-9 gel in a subject observed leakage from the introitus. A significant number of participants in a study conducted by Ramjee, et al., Aids Res. Ther., 4:20-29 (2007), on the delivery of 4 to 5 ml of Carraguard gel felt the gel caused excessive wetness.

It is therefore an object of the present invention to provide formulations and methods of administration that are effective in treating diseases and disorders of the female urogenital system, that also increase patient comfort and the likelihood that patients will follow their prescribed treatments.

It is a further object of the present invention to provide formulations and methods of administration that permit uptake of the drug in the affected area with minimal systemic side effects.

### BRIEF SUMMARY OF THE INVENTION

Drug delivery compositions which are suitable for transvaginal administration for the treatment of diseases and disorders of the urogenital tract are described. The drug delivery compositions are administered directly to the vagina using a convenient transvaginal application that deposits a very small volume of drug at the desired site for delivery. This method of administration reduces the systemic levels of the drugs and decreases the side effects which are associated with systemic administration. In the preferred embodiment, the compositions are in the form of a gel. The formulation is administered in volumes of less than or equal to 1 milliliter. In the preferred embodiment, the composition contains an antimuscarinc drug, such as oxybutynin.

### DETAILED DESCRIPTION OF THE INVENTION

### I. Drug Delivery Compositions

The drug delivery compositions consist of drug in combination with an excipient or polymeric carrier that controls and sustains release of drug over a period of time, at the site of transvaginal administration. The excipient or polymeric carrier may be used to alter the release rate (delay onset, increase rate of uptake, provide multiple releases) or to increase adhesion in the site of delivery (i.e., vaginal mucosa or epithelia). The drug formulation may be administered as a dried powder, solution, suppository, ovual, or aerosol, although in a preferred embodiment the drug is dissolved or suspended in a gel or polymer that is liquid or semi-solid at body and/or room temperature.

A. Drug

Drug refers to aqueous soluble drugs or micro- or nanoparticulates of non-soluble drugs, which achieve the desired effect. Drugs can be synthetic or natural organic compounds, proteins or peptides, oligonucleotides or nucleotides, or polysaccharides or sugars. Drugs may have any of a variety of activities, which may be inhibitory or stimulatory, such as antibiotic activity, antiviral activity, antifungal activity, steroidal activity, cytotoxic or anti-proliferative activity, anti-inflammatory activity, analgesic or anesthetic activity, or be useful as contrast or other diagnostic agents. In general, however, drugs are those having a local or regional effect, which typically does not include hormones or anti-cancer drugs requiring systemic levels of drug for efficacy. A description of classes of drugs and species within each class can be found in Martindale, The Extra Pharmacopoeia, 31st Ed., The Pharmaceutical Press, London (1996) and Goodman and Gilman, The Pharmacological Basis of Therapeutics, (9th Ed., McGraw-Hill Publishing company (1996)).

A preferred drug is an α-adrenergic agonist. The drug may be an antimuscarinic, such as DETROL® LA (tolterodine tartrate) (Pharmacia Corp.), propantheline, and oxybutynin hydrochloride.

B. Carriers and Excipients

The carrier may be an ointment, gel, paste, lotion, sponge, powder, or spray, soft gelatin capsules. The drug may be in a paste or gel which is placed in a soft gelatin capsule. Suitable excipients are known to those skilled in the art. See, for example, Remington's Pharmaceutical Sciences, 20th ed., Lippincott Williams & Wilkins, Baltimore, Md., 2000. As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, polyvinylpyrrolidone, sugars and starches.

Polymers can be used to increase adhesion to mucosal surfaces, to control release as a function of the diffusion rate of drugs out of the polymeric matrix and/or rate of degradation by hydrolysis or enzymatic degradation of the polymers and/or pH alteration, and to increase surface area of the drug relative to the size of the particle.

Gels have been used for vaginal delivery of many active agents, which include several currently marketed gels (reviewed in Neves, Int. J. Pharmaceuticals, 318:1-14 (2006); See also, Barnhart, et al., Contraception, 72:65-70 (2005)). WO 98/11888 discloses formulations including creams and gels, for intravaginal delivery of anti-incontinent agents. Several gels have been used for delivery of hormones in animals, such as described in U.S. Pat. No. 6,908,623 or marketed by GelMed Pharmaceuticals.

Controlled or sustained release formulations are typically prepared by modification or coating of the drug particles, which are then suspended in the carrier. These are well known. Delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et al., (Media, Pa.: Williams and Wilkins, 1995) which provides information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, particles may also contain minor amount of nontoxic auxiliary substances such as wetting or emulsifying agents, dyes, pH buffering agents, and preservatives.

Extended Release Dosage Forms

Extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

An immediate release portion can be added to the extended release system by means of either applying an immediate release layer on top of the extended release core using coating or compression process or in a multiple unit system containing extended and immediate release beads. Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

Delayed release formulations are created by coating a solid dosage form with a film of a polymer which is insoluble for a period of time in the environment in which release is to occur. The delayed release dosage units can be prepared, for example, by coating a drug or a drug-containing composition with a selected coating material. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

C. Dosage

The compositions are administered to a patient in an amount that delivers an effective amount of drug within the region to be treated, in this case the uro-genital region. Studies have demonstrated that despite vascular connections to the systemic vascular system, drugs administered transvaginally are primarily retained within the uro-genital region. In the case of oxybutynin, the preferred dosage in an amount equivalent to approximately four to twelve mg of drug, most preferably 4 mg per administration. In the preferred embodiment, the formulation contains a dosage in the low end of the approved range. The formulation is prepared so that the dose is delivered in a volume of less than or equal to one milliliter. The compositions may result in sustained, continuous release of the drug or immediate, i.e. burst, as well as immediate followed by sustained release of drug.

### II Methods of Using the Compositions

The drug delivery compositions are applied transvaginally. The result is uptake of the drug in the urogenital area. To treat incontinence, this occurs when the drug binds the bladder receptors in what has been designated the “first pass bladder effect”.

The formulations can be used in the treatment of infections such as infections of the urinary tract, urinary incontinence, urethral syndrome, urethritis, female sexual dysfunction, and interstitial cystitis.

The drug compositions are applied once or twice daily throughout the period that a patient has the disorder, infection, or disease. A volume of less than one milliliter is applied using an applicator. In the preferred embodiment, the applicator is pre-loaded and disposable. It contains a tip shaped and of a length suitable for ease of insertion into the vaginal vault, and includes a depressor that deposits through the tip the small amount of formulation, less than a milliliter, more preferably less than 500 microliters, most preferably 100 to 200 microliters, directly onto the vaginal vault wall. In a preferred embodiment, the application is pre-loaded with drug, disposable and provided in a sterile package with instructions for use.

For the treatment of urinary incontinence, the application of the formulations may result in increased bladder capacity and/or decreased Bladder Compliance. Bladder Compliance refers to the change in pressure per unit volume (ΔP/V). Thus a decrease in Bladder Compliance due to the administration of a drug, such as oxybutynin, results when the pressure is lower after the administration of the drug than it was prior to the administration of the drug for the same volume at micturition.

### EXAMPLES

### Example 1

### Clinical Studies

A 68 year old caucasian female developed urgency incontinence two years after her laparoscopically assisted transvaginal hysterectomy. The patient was then treated with DETROL® (2 mg) orally twice a day; and she experienced a slight improvement. Then, the medication was changed to Ditropan XL and Premarin transvaginal cream (1 gm) INSERT VOLUME. every night, and she noticed additional improvement. However she experienced side effects, such as dry mouth, from the drug. Two weeks later, she started applying oxybutynin (5 mg) into the vagina at bed time, instead of Ditropan XL. Her urgency symptoms improved remarkably, and she did not experience any noticeable side effects. The patient used transvaginal oxybutynin (5 mg) at bed time for 6 months. Throughout the treatment period, the drug remained effective at the lower dosage (5 mg) in controlling her symptoms of urgency incontinence, and she did not suffer from any side effects.

A 43 year old caucasian female with a history of chronic pelvic pain and multiple surgical procedures, including pain mapping under conscious sedation and repair of occult bilateral inguinal hernias, developed urinary frequency, urgency and nocturia. Her urine analysis and urine culture were negative for signs of urinary tract infection. Her treatment began with transvaginal oxybutynin (5 mg) once daily, at bed time, applied into the vagina. She noticed significant improvement. When the dose was increased to 5 mg of oxybutynin into the vagina twice daily, she noticed remarkable improvement of her symptoms and no significant side effects.

### Example 2

### Efficacy of Small Volumes of Oxybutynin

Administration of semi-solid vaginal preparations suffer from poor patient compliance attributed mainly to the messiness associated with leakage (influenced in part by large volume of material typically administered intravaginally).

A study was designed to evaluate the safety and therapeutic efficacy of intravaginal administration of a small volume of low dose oxybutynin, in subjects with urge urinary incontinence. Animal studies conducted in support of the treatment protocol demonstrated that an oxybutynin gel instilled into the vagina of rabbits over 14 days resulted in a significant decrease in micturition pressure and an increase in functional bladder capacity. The protocol (including changes to Protocol), Informed Consent, plans for advertising regarding subject recruitment, and subject stipend (and annual, if applicable) were approved by a properly constituted committee or review board (e.g., Institutional Review Board [IRB]) recognized by the US Food and Drug Administration (FDA) for approving studies in human subjects (21 CFR Part 56).

During the 2-week screening period, potential study subjects were required to complete a daily diary recording the frequency of micturition and urine leakage/incontinence episodes. Subjects returned to the site after completing two weeks of screening diary for determination of whether they met all inclusion/exclusion criteria. General inclusion/exclusion were as follows:

### Main Inclusion Criteria—

(1) Female between the ages of 18 to 75 years of age

(2) Had symptoms of urinary urgency with an increased frequency of micturition (a minimum of an average of at least 10 voids per 24 h.) and/or an average of at least 7 (but <28) episodes of urinary incontinence per week, as confirmed during each week of the two-week screening diary.

### Main Exclusion Criteria:

(1) Had taken in the 2 weeks prior to screening or anticipate taking at any time during the course of the study (screening through the end of study) the following prescription and/or OTC medications: Amantadine, Antidepressants, Antihistamines, Clozapine, Disopyramide, Domperidone, antimycotic agents, (e.g., ketoconazole), Levodopa, MAOIs, Metoclopramide, Nefopam, Nitrates, Parasympathomimetics, Phenothiazines, pseudophedrine, or macrolide antibiotics (e.g., erythromycin and clarithromycin).

(2) Had lower urinary tract surgery within 6 months prior to Screening.

(3) Had a diagnosis of interstitial cystitis, urethral syndrome, painful bladder syndrome, stress incontinence, overflow urinary incontinence, any urinary condition or disease which would in the investigator's opinion preclude the subject from participating in the study.

(4) had significant or unstable (in the opinion of the Investigator) neurological, cardiac, pulmonary, hepatic, renal, gastrointestinal, hematological, gynecological, endocrine, and/or metabolic disease or disorder.

### Safety, Efficacy and Compliance

Safety and efficacy were assessed throughout the study as follows. Safety parameters included physical examinations, ECG, Vital signs (sitting blood pressure [BP] and heart rate [HR]), PAP smears, Vaginal irritation inspections, Colposcopy, Clinical laboratory tests (hematology, blood chemistry, blood lipids, and urinalysis) and serum pregnancy tests, Plasma levels of oxybutynin and desethyloxybutynin, and assessment of adverse events (AE). The efficacy parameters included the number of micturitions and incontinence episodes recorded on the daily diary.

### Test Material

Oxybutynin HCl, USP (4-(diethylamino)-2-butynyl (±)-alpha-phenylcyclohexaneglycolate hydrochloride) for intravaginal use was supplied by FemmePharma Global Healthcare, Inc. in unit dose applicators. Oxybutynin was formulated in a unit volume of 0.2 mL.

### Vaginal Applicator

Each single-use, unit-dose vaginal applicator was designed to administer a small volume of oxybutynin. After receiving instruction on the use of the vaginal applicator (see Appendix IV), the subject was asked to administer their first treatment dose during their baseline visit. The test material (oxybutynin 4 mg in a total volume of 0.2 mL) was administered using a vaginal applicator, which. The applicator was designed to allow subjects to administer study drug high into the vaginal vault. Subjects were provided with instructions on how to apply the vaginal applicator. During each day of the 3-week treatment period, subjects administered their assigned number of applicators containing study drug at approximately the same time each morning. Subjects returned to the study clinic weekly for safety assessments and at Visit 7 (Week 4) for a post-treatment safety assessment.

### Daily Diary

Each day throughout the study, from Screening to the End-of-Treatment (Visit 6), the subjects kept a written daily diary. The daily diary was designed to capture the frequency of micturition and the number of episodes of urine leakage/incontinence. At each study visit, the subject returned the written daily diary for that previous treatment period to the study coordinator/study staff member who reviewed it for completeness and clarity. The daily diary captured treatment information, including the number of applicators used each day.

### Study Group

Fifteen women (mean age 49 years; range 29-61) were dosed with 0.2 ml Oxybutynin HCl intravaginal gel (4 mg/day) for 21 days. Subjects recorded in a diary for 14 days of screening prior to drug treatment and then during the 21 days of treatment the number of micturitions per day and the number of incontinence events. Based on these diary data the number of micturitions and incontinence events during the final seven days of screening were respectively 15.4±7.40 (mean±S.D.) micturitions/day and 1.2±1.4 accidents/day. These respectively decreased to 11.6±3.2 micturitions/day and 0.4±0.6 accidents per day during the third week on treatment. These decreases were both judged to be significantly significant (i.e. P-value<0.05) based on a paired t-test (two-tailed). Importantly, only 16 side-effects were reported. These are summarized in Table I.

TABLE I
Adverse Events

| Adverse Event | Number of Occurrences |
|---|---|
| Headache | 7 |
| Dizziness | 2 |
| Pyrexia | 2 |
| Neck pain | 1 |
| Pain in extremity | 1 |
| Abdominal pain | 1 |
| Nasopharyngitis | 1 |
| Dysmenorrhea | 1 |

Typical anti-muscarinic side-effects (e.g. dry-mouth) were neither observed nor reported in the studied population.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

We claim:

1. A transtransvaginal drug formulation comprising
a drug for relief of a disease or disorder of the uro-genital region of a female,
in a pharmaceutically acceptable carrier for administration to the vagina,
wherein the drug is in the formulation in an amount effective to provide relief from diseases or disorders of the urogenital system, and
wherein the formulation is in a volume of one milliliter or less.

2. The drug formulation of claim 1 wherein the drug is in the form of micro- or nano-particulates suspended in a pharmaceutically acceptable carrier.

3. The drug formulation of claim 1 wherein the drug is soluble in aqueous solutions.

4. The drug formulation of claim 1 wherein the drug is insoluble in aqueous solutions and.

5. The drug formulation of claim 1 wherein the carrier is selected from the group consisting of a solution, dry powder, ointment, cream, foam, semi-solid suppository, ovual, or aerosol.

6. The drug formulation of claim 1 wherein the drug is selected from the group consisting of α-adrenergic agonists and antimuscarinics.

7. The drug formulation of claim 1 wherein the drug is a steroid selected from the group consisting of progestins, estrogens, antiestrogens, and antiprogestins.

8. The drug formulation of claim 6 wherein the drug is an antimuscarinic selected from the group consisting of tolterodine tartrate, propantheline, and oxybutynin hydrochloride.

9. The drug formulation of claim 8 wherein the drug is oxybutynin hydrochloride and the excipient is a gel.

10. The drug formulation of claim 9 wherein the formulation is in a volume of less than one milliliter and delivers between 4 and 12 mg per administration.

11. The drug formulation of claim 10 wherein the formulation is in a volume of 200 to 500 microliters.

12. The drug formulation wherein the formulation provides controlled or sustained release of the drug.

13. A method for treating a disease or disorder of the urogenital system comprising
administering to the vagina the drug formulation of any of claims 1-12.

14. The method of claim 13 administered to a patient in need of treatment for urinary incontinence.

15. The method of claim 13 wherein the drug is administered twice daily.

16. An applicator for transvaginal administration of a drug formulation, comprising the formulation of claim 1.

17. The application of claim 16 that is disposable and provided in a sterile package with instructions for use.
